(19) United States
(12) Patent Application Publication
(10) Pub. No.: US 2020/0185095 A1
(43) Pub. Date: Jun. 11, 2020

(54) PLUG-AND-PLAY MEDICAL INTEROPERABILITY AND DATA LIQUIDITY PLATFORM

(71) Applicant: CENTER FOR MEDICAL INTEROPERABILITY INC., Nashville, TN (US)

(72) Inventors: EDWARD MICHAEL MILLER, FRANKLIN, TN (US); WILLIAM SPENCER CROSSWY, BRENTWOOD, TN (US); SUMATH CHANNABASAPPA, BROOMFIELD, CO (US)

(21) Appl. No.: 16/706,462

(22) Filed: Dec. 6, 2019

Related U.S. Application Data
(60) Provisional application No. 62/776,289, filed on Dec. 6, 2018.

(51) Int. Cl. G16H 40/67; G06F 9/54; G16H 40/40; H04L 29/08

(52) U.S. Cl. CPC G16H 40/67; H04L 67/125; G16H 40/40; G06F 9/542

(57) ABSTRACT

A system or platform with architecture for dynamically orchestrating secure data flows across components in a healthcare enterprise, including, but not limited to, medical devices, electronic health records, clinical decision support systems, patient administration systems, and the like. The present invention enables plug-and-play interoperability via standards-based, trusted, bi-directional communication between components. The system provides dynamic registration of components and their profiles, runtime provisioning of communications channels, and dynamic orchestration of data between components. Component profiles abstract devices and applications as services producing and consuming certain messages that represent portions of a canonical data model. Registration takes a component identity and assigns an identifier and address to broker communication with the component. Dynamic orchestration uses registered component profiles and workflows to dynamically provision communication channels and initiate and manage data flow between components. The system also provides a process to remotely control medical devices via automated or semiautomated processes.

This application claims benefit of and priority to U.S. Provisional Application No. 62/776,298, filed Dec. 6, 2018, and is entitled to that filing date for priority. The specification, figures, and complete disclosure of U.S. Provisional Application No. 62/776,298 are incorporated herein in their entireties by specific reference for all purposes.

### FIELD OF INVENTION

The present invention relates to a system and related methods for coordinating secure data flows across and between components in a healthcare enterprise, enabling plug-and-play interoperability and remote control and direction of certain components, such as, but not limited to, medical device or other modalities.

### BACKGROUND OF THE INVENTION

The current state of prior art healthcare systems includes non-standard data interfaces with static provisioning of data flows. This impedes care delivery, the management of technical assets, and the efficient and timely implementation of clinical workflows. As the healthcare industry moves toward person-centered care, the current state also limits innovation.

In particular, a great variety of medical devices produce data about patients to aid in their immediate and long-term care, but the device interfaces are not standardized, vary widely from device to device, and largely remain proprietary. As a result, health care systems must employ additional products or services to address the issue, often at significant cost.

### SUMMARY OF INVENTION

In various embodiments, the present invention comprises a system or platform with architecture for dynamically orchestrating secure data flows across components in a healthcare enterprise, including, but not limited to, medical devices, electronic health records, clinical decision support systems, patient administration systems, and the like. The present invention enables plug-and-play interoperability via standards-based, trusted, bi-directional communication between components. Additionally, it provides for and allows an efficient process to remotely control medical devices via automated or semiautomated processes.

### BRIEF DESCRIPTION OF THE DRAWINGS

FIG. 1 a diagram of a system platform in accordance with an exemplary embodiment of the present invention.

FIG. 2 is a diagram of an example of a workflow management process.

FIG. 3 is diagram of the architectural role of the canonical data model in the system of FIG. 1.

FIG. 4 is a diagram illustrating a process for component registration and dynamic provisioning of topics.

FIG. 5 is a diagram illustrating various system platform events triggering the orchestration engine to create or modify communications channels, using topics as an example of such channels.

### DETAILED DESCRIPTION OF EXEMPLARY EMBODIMENTS

In various embodiments, the present invention comprises a system or platform with architecture for dynamically orchestrating secure data flows across components in a healthcare enterprise, including, but not limited to, medical devices, electronic health records, clinical decision support systems, patient administration systems, and the like. The present invention enables plug-and-play interoperability via standards-based, trusted, bi-directional communication between components. Additionally, it provides for and allows an efficient process to remotely control medical devices via automated or semiautomated processes.

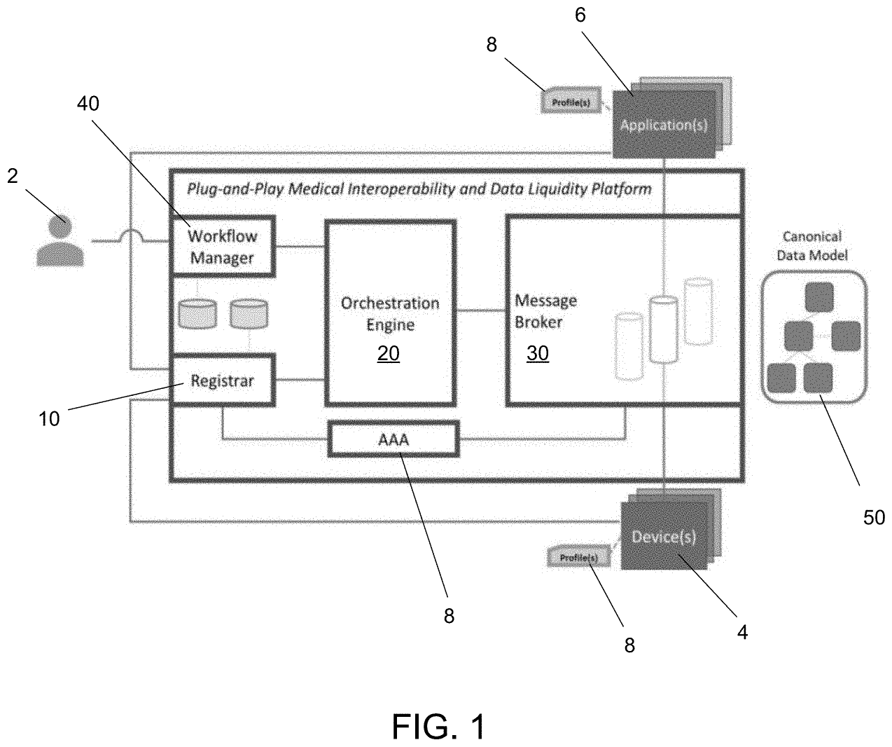

In several embodiments, as seen in FIG. 1, components such as devices 4, applications 6, or other forms of clients connect and register through a registrar 10 application or component with an identity and a set of capabilities through a control channel that is either known a priori or discovered. The registration process assigns an identifier and a network address through a namespace that allows the orchestration engine 20 to broker communications (such as through a message broker 30) with the component (e.g., device, application, or the like). The key functions and actors comprising the platform are described below.

Components include, but are not limited to, devices 4 (e.g., point-of-care medical devices), applications 6 (e.g., EMRs, analytic apps), or other platforms or systems that can connect to and communicate via the system platform. An extensible set of predefined component classes (e.g., “vitals monitor”, “ventilator”, “alerting system”) may provide a simple abstraction for categorizing components.

Component profiles 8 are the mechanism for components 4, 6 to register their identity and capabilities. Capabilities registered include, but are not limited to, the data produced or consumed by the component, such as, but not limited to, published messages, message subscriptions, and receivable commands. The profile may also include metadata, such as, but not limited to, the component's name, component type, vendor name, version, and component class. The profile itself may be implemented using formats such as JSON, XML, or the like, which can be used to communicate the necessary set of information.

A component registrar 10 is responsible for registering components when they connect to the system platform, and de-registering or unregistering a component upon disconnect. The registrar also provides component naming and discovery services, such as, but not limited to, assigning an identifier and network address to components when they connect, and sharing the identifier, address, and capabilities with the orchestration engine 20 and message broker 30. Registrar implementation may be through software and technologies such as, but not limited to, DNS, with extensions, modifications or additions necessary to implement the functions described herein.

The message broker 30 supports and manages routing and delivering of messages between components. Implementation of the message broker may be through technology such as, but not limited to, Apache Kafka, AMQ, DDS, and the like, with extensions, modifications or additions necessary to implement the functions described herein.

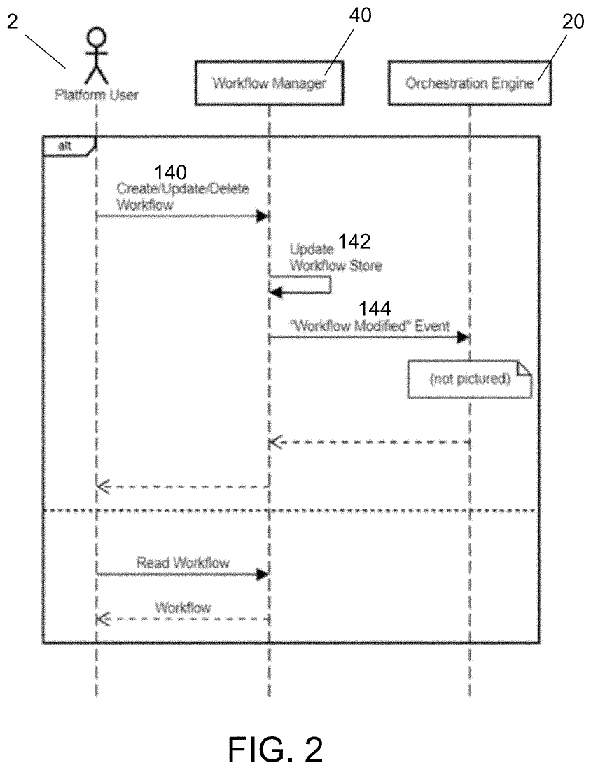

The workflow manager 40 supports and manages the creating, editing, and querying of business rules, workflows, and processes. Workflow management may be implemented with business process modeling technologies, with extensions, modifications or additions necessary to implement the functions described herein. FIG. 2 illustrates an example of as scenario in which a user 2 creates, updates, deletes or otherwise modifies 140 workflows. The system then updates the workflow store 142 in the workflow manager, which forwards the “workflow modified” event 144 to the orchestration engine 20.

In several embodiments, a “pre-packaged” or established standard core set of messages and workflows appropriate for the particular domain or environment may be provided. For example, this would include certain messages and workflows related to software update and management of medical devices that are common and valuable enough for inclusion in any implementation of the system platform. Any set of workflows and messages is extensible for evolution and local needs. “Default” workflows also may be provided or specified, such that messages or events with no workflows otherwise specified or applicable to those messages or events, are handled in a way that provides baseline utility (e.g., logging and storing the original message).

The orchestration engine 20 consumes messages from components and uses defined workflows and registered components' profiles to dynamically provision communication channels and route those messages to appropriate destinations, usually in conjunction or through the message broker 30. This allows business processes to be implemented by a dynamic orchestration of data flow between components connected to the system platform and the contextualization of data. This is in stark contrast to the prior art, where communication channels between components are statically provisioned (i.e., fixed) with little or no context. Dynamic orchestration may be enabled through business process modeling languages and data broker technologies, along with component profiles, thereby enabling plug-and-play communication and dynamic implementation of clinical workflows.

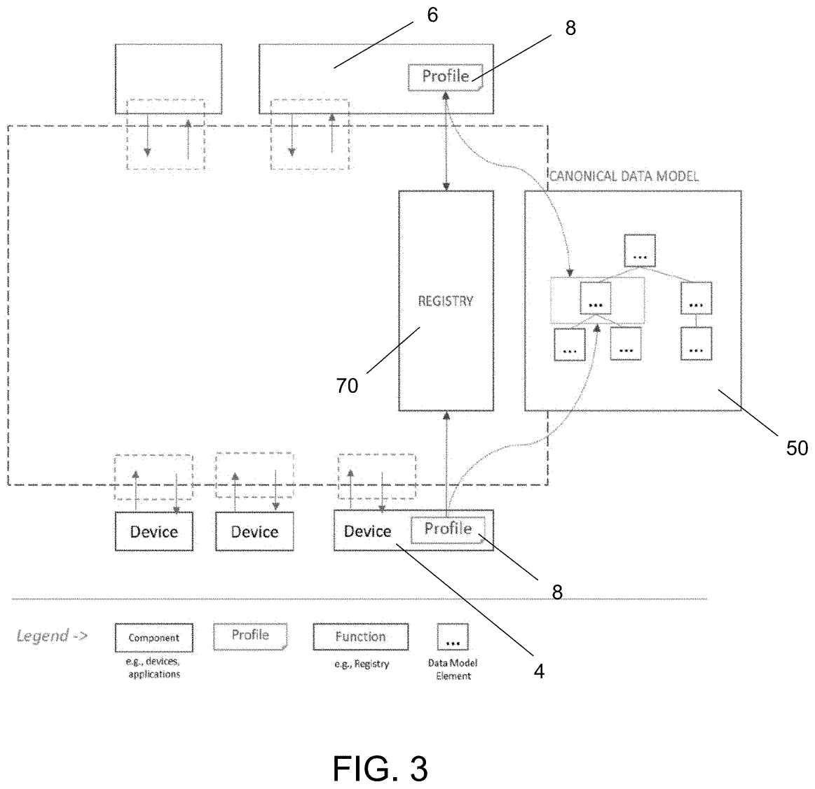

A Canonical Data Model (CDM) 50 describes all possible data communicated via the platform. The CDM may include, but is not limited to, a collection of interoperable data models, terminologies and definitions, and related constructs, that are shared by a collection of at least some, or all, of the components in the system platform, and are used to describe the data produced and/or consumed by one or more of the components. This allows the data exchanged by components to be “known,” and sharing this known data over standard interfaces enables interoperable data exchange and orchestration. In some embodiments, a core set of domain-appropriate and/or standard constructs may be provided or “pre-packaged,” but can be extended or modified for local needs and evolution. Components' profiles include the types of messages they produce or consume in terms of what portion of the CDM 50 each message represents. Components document the data they exchange by referencing elements of the applicable CDM, and the interface or mechanism for exchanging that data, in their respective profiles (as described above, these profiles are shared during component registration as part of establishing communications with the system platform). This enables the orchestration engine 20 to analyze and reason over the relationship between components' produced and consumed data, and thereby appropriately provision and broker communications between or involving various components, such as, but not limited, in response to various events. An example of this communication process is shown in FIG. 3.

Messages may be grouped into categories such as “commands” and “events.” A set of pre-defined, standardized messages enable a certain degree of off-the-shelf interoperability, but the set of pre-defined messages is extensible and may be added to or modified to accommodate local needs and evolution. Examples of pre-defined events in the medical domain include, but are not limited to, “Patient Admission Event”, “Vitals Observation Event”, and the like. Examples of pre-defined commands include, but are not limited to, “Update Device Software”, “Report Patient Vitals”, and the like. All inbound messages undergo appropriate validation, parsing, normalization (e.g., converted to a standard format, if needed), and contextualization to ensure that any message is well-formed and in alignment with the canonical data model.

Communications are conducted under an authentication, authorization, and auditing (AAA) framework 60. A security infrastructure (such as a public key infrastructure) enables authentication and trusted communication with and between components. Permissions-based authorization supports allowing or restricting a component's ability to connect to the system platform and produce or consume messages. Accounting functions support configurable logging and querying of platform events. Configuration may include, but is not limited to, event types, logging levels, log persistence duration, and similar parameters.

The system provides for dynamic communication of component profiles. Component profiles may change over time due to software or hardware updates that impact a component's functionality. These profile changes may dictate a new registration or registration update, which then dictate actions taken by the orchestration engine, as described below.

The system also provides for dynamic provisioning of communication channels, as described above. To enable dynamic data flow between components connected to the Platform, workflows and components' capabilities dictate the runtime creation and deletion of communication channels for components to write to or read from. A communication channel is any mechanism by which data “sent” from one component can be “received” by one or more other components. This may be implemented in a publish-subscribe model with technologies that use the notion of a “topic” to abstract an underlying data transport mechanism such as a file system for the purpose of exchanging a particular type of data. Other implementations may leverage message routers, networking technologies such as multicast groups, shared memory architectures, or many other different approaches. Throughout this document, a “topic”-based publish-subscribe model is used to describe an implementation of communications channels and their dynamic provisioning, but this is not a required form of implementation, and other forms of communications channels implementation are within the scope of this invention. In a publish-subscribe model based implementation, components publish and subscribe to topics that are created by the Orchestration Engine on demand. Topics that are no longer needed may be destroyed. This system is effectively a runtime Create, Read, Update, Delete (CRUD) interface for topics in which components' needs to communicate dictate the Creating or Deleting of topics that those components then Update or Read from. Topic names may be automatically generated by the Orchestration Engine, or components may specify a topic name with any conflicts handled by the Orchestration Engine. Other naming techniques may also be employed.

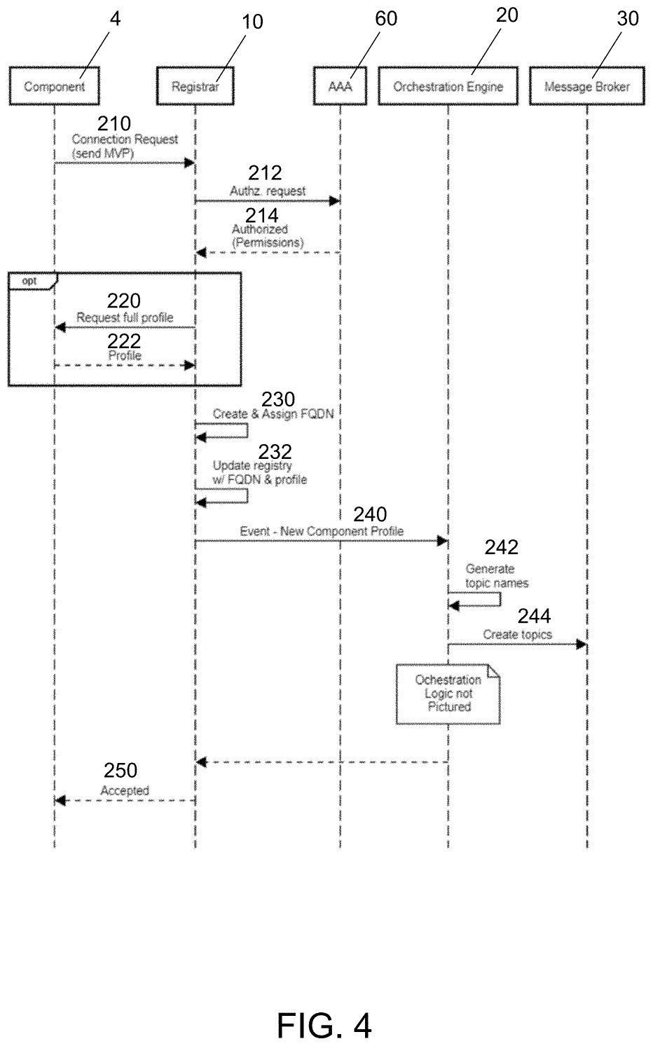

FIG. 4 illustrates an example of a component 4 connecting to the system platform for the first time, and the orchestration engine 20 using the component's profile 8 to dynamically create communications channels (using topics as an example of such channels). The component sends a connection request 210 to the system platform registrar 10, which seeks 212 and obtains authorization 214 from the AAA framework 60. Upon receiving authorization, the registrar requests the full profile 220 from the component 4, which sends 222 the profile to the registrar. The registrar creates and assigns 230 a fully qualified domain name (FQDN) to identify and specify the location the component, and updates 232 the registry with that information and stores the profile info. An event message 240 for a new component profile is sent to the orchestration engine 20, which generates topic names 242 and directs the message broker to create topics 244. Upon completion of this work by the orchestration engine, messages are sent back to the registrar, which sends a message of acceptance 250 to the component.

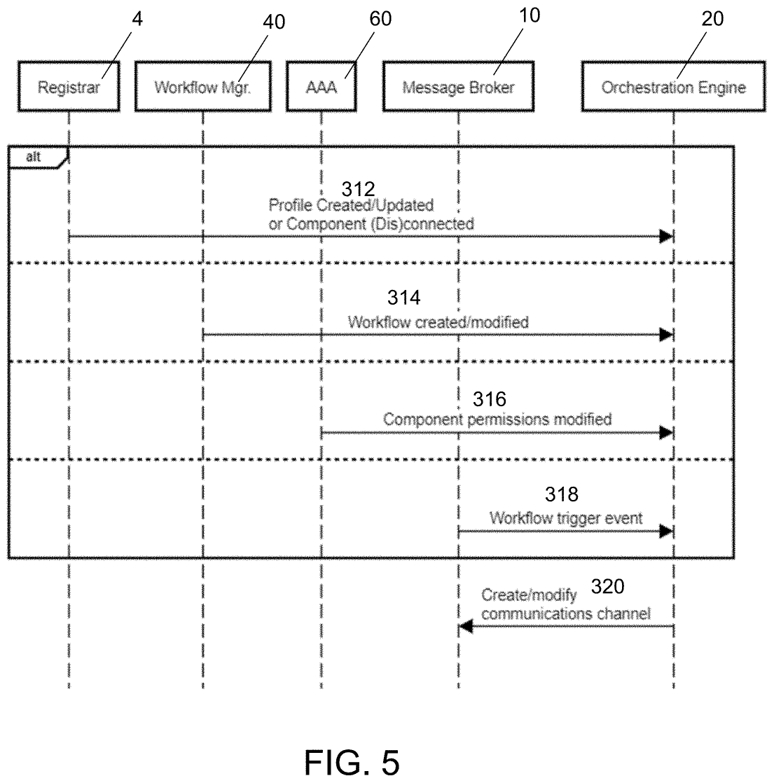

A variety of platform events could trigger this provisioning, including component profile registration or update, workflow creation or modification, or permissions modification. An example of these events and their effects on communications channel provisioning 320 are shown in FIG. 5. Events include, but are not limited to, a profile being created or updated or a component being connected or disconnected 312, a workflow being created or modified 314, component permissions being modified 316, or a workflow triggering event 318.

The following examples illustrate how the present invention enables the dynamic integration of medical devices and components into a hospital healthcare information system:

### EXAMPLE 1

A hospital would like to remotely update software on medical devices. The present invention enables this process to be implemented in the following way:

1. A device management entity registers with the platform. Its profile indicates it can send “Update Software” commands and receive “Software Update Status” events. The registrar assigns an identifier and network address to the device management entity and shares this information with the orchestration engine, which provisions the appropriate communication channels.

2. A medical device registers with the platform. Its profile indicates it can receive “Update Software” commands and send “Software Update Status” events. The registrar assigns an identifier and network address to the device management entity and shares this information with the orchestration engine, which provisions the appropriate communication channels.

3. A user defines a workflow, such as: “When a management entity sends an ‘Update Software’ command indicating a particular device should update its software, the appropriate device receives the command, attempts to update its software, and then sends a ‘Software Update Status’ event back to the management entity that initiated the update to any medical device.”

4. The orchestration engine has the message broker dynamically provision a communication channel between the medical device and management entity, so the device and entity can send the appropriate software update events to one another and implement the defined workflow.

### EXAMPLE 2

A hospital would like to set a policy that any physiologic monitoring device (“vitals monitor”) associated with a patient should send data to a monitoring application. The present invention enables the hospital to implement this business process in the following way:

1. A user defines a workflow, such as “Any vitals device associated with any patient sends its data to ‘Vitals App’.”

2. A vitals device registers with the system platform. The device profile indicates it produces vitals-signs data.

3. ‘Vitals App’ (an application component) registers with the system platform. The application profile indicates it consumes vitals-signs data.

4. The vitals device sends a “Patient Device Association” event to the system platform.

5. The orchestration engine receives the event, checks the workflow manager and finds the workflow from Step 1 should be triggered, checks the component registrar and finds the vitals device and monitoring app should participate in the workflow, commands the message broker to dynamically provision a communication channel to route the device's vitals data to the vitals monitoring application.

In one exemplary embodiment, the present invention, and particularly the CDM element, may incorporate content and/or resources consistent with the Fast Healthcare Interoperability Resources (FHIR) specification. Core clinical data resources would provide starting definitions for clinical data exchange. Constrained FHIR resources coupled with terminology value sets may be used to describe specific types of clinical data, e.g., physiological monitoring observation data. Events, commands, and/or infrastructure data and messages exchanged by components (e.g., registration message, updating profile information) may also be described at least in part with FHIR resources.

The CDM describes what data is shared, while mechanisms for how that data is shared (i.e., over what interface or exchange mechanism) are part of the capability of a component, which is captured in its component profile, as described above. For an FHIR-related implementation, RESTful FHIR APIs may be used as a method of data exchange, along with other exchange mechanisms (e.g., THE PCD transactions, HL7 2.6 messages for point-of-care devices). Unambiguous specification for how FHIR resources are exchanged by all mechanisms may be specified as part of the CDM.

Similarly, the CDM needs various ways or methods of being authored (created), maintained, and shared. For an FHIR-related implementation, all of the interfaces and exchange mechanisms, the data models describing data exchanged over those interfaces, and all related artifacts (e.g., FHIR resources, profiles, valuesets, extensions, mappings, and the like) may be hosted in or referenced on an authoritative interface and data model registry 70. The registry contents may be vetted and approved by an appropriate governing body, following a well-defined curation process to maintain interoperability, ensure quality, and manage evolution and versioning.

In general, as described above, the CDM should be extensible to accommodate local needs, variations in implementations, and evolution of the model. For an FHIR-related implementation, the extensibility mechanisms may include FHIR-related mechanisms. For example, a component supporting FHIR resources for reporting physiological monitoring observations may comply with the base resource definitions, and also report additional observation types using other vocabularies, including but not limited to private or proprietary vocabularies. The CDM thus specifies the data models most crucial for interoperability, while still allowing and supporting flexibility and innovation.

Similarly, for component registration, the registration function may be implemented with an FHIR API using an FHIR resource. FHIR APIs supported by the component may be known through the registration function through an FHIR CapabilityStatement.

Likewise, workflows may be implemented as business process models or business rules that use FHIR resources to specify data inputs and outputs. By leveraging profiles of components shared during registration (which includes supported FHIR resources), the orchestration engine may implement workflows using the known data expchanged by those components. For example, with reference to the business rule in Example 2 above, if the “vitals monitor connected” or “patient-device association” events are described using an FHIR resource, and used to specify the initial trigger for the business rule, then instances of that FHIR resource detected at runtime can trigger the workflow. Another example of a workflow may be “Periodically send observation data from this vitals monitor to an on-call physician.” This highlights particular types of data that would be captured in the CDM for this workout, including the following: vitals observations data; people (patients, physicians);

and alerts.

While the above description and examples are directed to a healthcare system platform and the integration of medical device data and communications, the system platform may be used for other, non-healthcare systems.

In order to provide a context for the various aspects of the invention, the following discussion provides a brief, general description of a suitable computing environment in which the various aspects of the present invention may be implemented. A computing system environment is one example of a suitable computing environment, but is not intended to suggest any limitation as to the scope of use or functionality of the invention. A computing environment may contain any one or combination of components discussed below, and may contain additional components, or some of the illustrated components may be absent. Various embodiments of the invention are operational with numerous general purpose or special purpose computing systems, environments or configurations. Examples of computing systems, environments, or configurations that may be suitable for use with various embodiments of the invention include, but are not limited to, personal computers, laptop computers, computer servers, computer notebooks, hand-held devices, microprocessor-based systems, multiprocessor systems, TV set-top boxes and devices, programmable consumer electronics, cell phones, personal digital assistants (PDAs), network PCs, minicomputers, mainframe computers, embedded systems, distributed computing environments, and the like.

Embodiments of the invention may be implemented in the form of computer-executable instructions, such as program code or program modules, being executed by a computer or computing device. Program code or modules may include programs, objects, components, data elements and structures, routines, subroutines, functions and the like. These are used to perform or implement particular tasks or functions. Embodiments of the invention also may be implemented in distributed computing environments. In such environments, tasks are performed by remote processing devices linked via a communications network or other data transmission medium, and data and program code or modules may be located in both local and remote computer storage media including memory storage devices.

In one embodiment, a computer system comprises multiple client devices in communication with at least one server device through or over a network. In various embodiments, the network may comprise the Internet, an intranet, Wide Area Network (WAN), or Local Area Network (LAN). It should be noted that many of the methods of the present invention are operable within a single computing device.

A client device may be any type of processor-based platform that is connected to a network and that interacts with one or more application programs. The client devices each comprise a computer-readable medium in the form of volatile and/or nonvolatile memory such as read only memory (ROM) and random access memory (RAM) in communication with a processor. The processor executes computer-executable program instructions stored in memory. Examples of such processors include, but are not limited to, microprocessors, ASICs, and the like.

Client devices may further comprise computer-readable media in communication with the processor, said media storing program code, modules and instructions that, when executed by the processor, cause the processor to execute the program and perform the steps described herein. Computer readable media can be any available media that can be accessed by computer or computing device and includes both volatile and nonvolatile media, and removable and non-removable media. Computer-readable media may further comprise computer storage media and communication media. Computer storage media comprises media for storage of information, such as computer readable instructions, data, data structures, or program code or modules. Examples of computer-readable media include, but are not limited to, any electronic, optical, magnetic, or other storage or transmission device, a floppy disk, hard disk drive, CD-ROM, DVD, magnetic disk, memory chip, ROM, RAM, EEPROM, flash memory or other memory technology, an ASIC, a configured processor, CDROM, DVD or other optical disk storage, magnetic cassettes, magnetic tape, magnetic disk storage or other magnetic storage devices, or any other medium from which a computer processor can read instructions or that can store desired information. Communication media comprises media that may transmit or carry instructions to a computer, including, but not limited to, a router, private or public network, wired network, direct wired connection, wireless network, other wireless media (such as acoustic, RF, infrared, or the like) or other transmission device or channel. This may include computer readable instructions, data structures, program modules or other data in a modulated data signal such as a carrier wave or other transport mechanism. Said transmission may be wired, wireless, or both. Combinations of any of the above should also be included within the scope of computer readable media. The instructions may comprise code from any computer-programming language, including, for example, C, C++, C#, Visual Basic, Java, and the like.

Components of a general purpose client or computing device may further include a system bus that connects various system components, including the memory and processor. A system bus may be any of several types of bus structures, including, but not limited to, a memory bus or memory controller, a peripheral bus, and a local bus using any of a variety of bus architectures. Such architectures include, but are not limited to, Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computing and client devices also may include a basic input/output system (BIOS), which contains the basic routines that help to transfer information between elements within a computer, such as during start-up. BIOS typically is stored in ROM. In contrast, RANI typically contains data or program code or modules that are accessible to or presently being operated on by processor, such as, but not limited to, the operating system, application program, and data.

Client devices also may comprise a variety of other internal or external components, such as a monitor or display, a keyboard, a mouse, a trackball, a pointing device, touch pad, microphone, joystick, satellite dish, scanner, a disk drive, a CD-ROM or DVD drive, or other input or output devices. These and other devices are typically connected to the processor through a user input interface coupled to the system bus, but may be connected by other interface and bus structures, such as a parallel port, serial port, game port or a universal serial bus (USB). A monitor or other type of display device is typically connected to the system bus via a video interface. In addition to the monitor, client devices may also include other peripheral output devices such as speakers and printer, which may be connected through an output peripheral interface.

Client devices may operate on any operating system capable of supporting an application of the type disclosed herein. Client devices also may support a browser or browser-enabled application. Examples of client devices include, but are not limited to, personal computers, laptop computers, personal digital assistants, computer notebooks, hand-held devices, cellular phones, mobile phones, smart phones, pagers, digital tablets, Internet appliances, and other processor-based devices. Users may communicate with each other, and with other systems, networks, and devices, over the network through the respective client devices.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A system for managing electronic communications among medical care devices, comprising:
a plurality of distributed healthcare components, said components comprising at least one medical care device and at least one health application program;
a computer-based system platform comprising a plurality of functional elements and at least one computer server with a processor and non-transient data storage, said at least one computer server remotely located from at one of said plurality of distributed healthcare components;
wherein said plurality of functional elements comprise:
a registrar;
a message broker;
an orchestration engine; and
a canonical data model;
wherein the system platform stores a separate component profile for each of said plurality of distributed healthcare components that has registered with the system platform, said component profile identifying the types of messages produced or consumed by the associated component;
wherein, upon receiving an electronic message from a first distributed healthcare component, the orchestration engine automatically and dynamically provisions at least one communication channel from the first distributed healthcare component to a second distributed healthcare component based upon the component profile associated with the first distributed healthcare component and the component profile associated with the second distributed healthcare component.

2. The system of claim 1, wherein the canonical data model comprises a collection of interoperable data models, terminologies, and related constructs that are shared by at least some of the plurality of distributed healthcare components.

3. The system of claim 2, wherein said component profiles further describe the messages produced or consumed with reference to a portion of the canonical data model.

4. The system of claim 1, wherein transmission of the electronic message from the from the first distributed healthcare component to a second distributed healthcare component is performed by the message broker.

5. The system of claim 1, where the electronic message from the first distributed healthcare component comprises a command or an event.

6. The system of claim 5, wherein command messages are directed to the second healthcare component.

7. The system of claim 6, wherein the command message requests the second healthcare component to provide a response message with data or information.

8. The system of claim 6, wherein the command message requests the second healthcare component to update software.

9. The system of claim 5, wherein event messages describe or report an event.

10. The system of claim 8, wherein the event comprises patient admission, or data or testing observation.

11. The system of claim 1, wherein said plurality of functional elements further comprise:
a workflow manager.

12. The system of claim 1, wherein the electronic message is validated, parsed, and converted to a standard format if not in a standard format.

13. The system of claim 1, wherein component profiles are updated dynamically.
